(19) United States
(12) Patent Application Publication
(10) Pub. No.: US 2026/0158077 A1
(43) Pub. Date: Jun. 11, 2026

(54) Immature Pluripotent Fat Adult Stem Cell to Treat Multiple Human Diseases

(71) Applicant: Christina Bertolotto, Los Angeles, CA (US)

(72) Inventor: Christina Bertolotto, Los Angeles, CA (US)

(21) Appl. No.: 19/410,563

(22) Filed: Dec. 5, 2025

Related U.S. Application Data
(60) Provisional application No. 63/728,297, filed on Dec. 5, 2024.

(51) Int. Cl. A61K 35/545

(52) U.S. Cl. CPC A61K 35/545

(57) ABSTRACT

Extraction of a novel Immature Pluripotent Stem Cells (ImPS) to treat multiple diseases and treat patients with autologous or heterologous cellular therapies in multiple diseases.

### CROSS-REFERENCE TO RELATED APPLICATION

This application claims the benefit of U.S. Provisional Patent Application No. 63/728,297, filed on Dec. 5, 2024, the entire contents of which are incorporated herein by reference.

### BACKGROUND

Amyotrophic Lateral Sclerosis (ALS) is a devastating neurodegenerative and autoimmune disease that until today there has been no cure for. Currently, there are multiple clinical trials with different drugs and stem cell treatments trying to find a cure for ALS. ALS is a disease that attacks the central nervous system, specifically motoneurons and myelin. These cells are going through apoptosis, and rapid demyelination occurs. This translates into the patients as a loss of muscles and weight in a short period of time. The patients lose the capability to control movements. Usually, when this disease starts, it is more prominent on one side of the body. As the disease progresses, all muscles are compromised.

Accordingly, there is a need for an effective treatment for ALS.

### SUMMARY

The present subject matter relates to methods of obtaining human stem cells from adult adipose tissue. Other stem cells may be obtained from umbilical cord, placenta, amniotic membrane, and pancreatic islets of Langerhans. Once obtained, these human stem cells may be useful for a variety of indications, including but not limited to neurogenesis, neurodevelopment, neuroregeneration, molecular neurobiology, neurodegenerative diseases, the aging process, osteoarthritis, autoimmune diseases, lung diseases, cancers, and various other conditions.

The purpose of the present subject matter is to extract novel Immature Pluripotent Stem Cells (ImPS) to treat multiple diseases and treat patients with autologous or heterologous cellular products in multiple diseases.

### DETAILED DESCRIPTION

During a brief medical procedure, the therapeutic human stem cell product can be produced by a physician and clinical staff following a strict protocol.

The common name of a first product intended herein is Immature Pluripotent Stem Cells (ImPS). A first source of human cell, tissue, and cellular and tissue-based product (HCT/P) is fat tissue.

Adipose, or fat tissue, is composed of adipocytes, each innervated by a single capillary, with a background of white blood cells (resident macrophages) and progenitor/stem cells. As adipose is ubiquitous and relatively inert, it serves as a local regenerative bank for most major systems in the body, providing regenerative stem cells in times of injury or stress.

As described herein, the present subject matter relates to new methods for obtaining novel stem cells termed “Immature Pluripotent Stem Cells” or ImPS. Briefly, these tiny Immature Pluripotent Stem Cells (ImPS) have the following characteristics:
1. ImPS are present in adipose. Fat tissue, or adipose, is ubiquitous in soft tissues and surrounds major organs of the body. Relative to other tissues in the body, adipose is mostly inert, but highly vascularized and metabolically/endocrinologically active in times of stress.
2. It is possible to isolate ImPS via physical separation, without enzymes or chemical additives.
3. Typical of other stem cells, the amount of ImPS extracted varies from patient to patient.
4. When injected intravenously these cells can cross the blood-brain-barrier.

### ImPS-Specific Features

5. ImPS can differentiate to all three tissue types; endoderm, mesoderm, or ectoderm.
6. ImPS express classic stem cell markers (OCT3/4, SOX2, NANOG, Nestin, CD34, CD105, CD146).
7. Once cells cluster, they express a set cluster of differentiation markers (i.e. cell fate is fixed).
8. These cells can phagocytize (“eat”) other cells. Typically seen as a characteristic of immune cells (macrophages, monocytes, dendritic cells, etc.), the cells with greatest ability for phagocytizing other cells are actually reported to be embryonic stem cells.
9. These cells communicate and act through chemokine signals, formation of dendritic-like spines or processes, gap junctions (electric impulses), and through phagocytic incorporation of cellular components.

### Unique Defining Features of ImPS

10. Uniquely, the ImPS are much smaller than typical stem cells (1 micron); for reference, a red blood cell is 7 microns, an embryonic stem cell is 12-13 microns, and a normal fibroblast is 15-20 microns.
   a. Cells this size were documented by Doctors Bertolotto and Heneidi in 2005.
   b. First termed in 2006, these were not published widely in Scientific Literature until 2012, termed “Very small embryonic-like stem cells (VSELs)”. VSELs were poorly characterized and not from the same source as the cells described and obtained herein, with research using flow cytometric technologies including fluorescence-activated cells (FACS). These cells are described as responding to chemokine signals to go from the blood directly to sites of injury.
   c. These are not used in clinical applications and cannot be isolated without more complicated techniques and cell cultures.
11. UNIQUELY, the ImPS described and obtained herein do not need to be cultured.
   a. This trait is key for allowing a SINGLE DAY clinical procedure.
   b. Other stem cell isolates often require 1-2 WEEKS of culture, especially true of cells with the ability to differentiate to multiple cell types.
      i. Cultures will grow stem cell spheres, where clusters grow much in the same way an embryo is often depicted. This growth requires cell divisions and can only be done by extended cultures.
   c. The present process utilizes a unique property of ImPS (perhaps the tiniest of human cells), which is the ability to differentiate without forming a stem cell sphere or spherule.
   d. While not reported, the studies as described herein have observed that these cells are drawn to a specific cell type (typically injured cell, senescent cell, or cell in direct contact), and phagocytize the cell of choice.
   e. Once dozens of ImPS have eaten the cell of choice, they can form a cluster, rather than mitotically divide like cultures. This cluster can quickly differentiate (<48 hours) to the cell that the ImPS consumed. Interestingly, the nucleus of the cell is often still intact, indicating that nuclear genetic information is not what drives cellular division. Other research has shown similar phenomena, described in cancers, and never utilized for therapeutic purposes. Lastly, while the phenomenon is described in cancers, it is not linked to cancers; rather studies closely following cell fate and cell origin observed a similar phenomenon.

Accordingly, the ImPS are among the smallest stem cells that have been described. In certain embodiments, the ImPS can have a size of about 1 micron to about 10 microns. In certain embodiments, the ImPS nucleus can occupy the majority of the body of the stem cell, with a very small cytoplasm surrounding the nucleus of the ImPS. These stem cells can have a large amount of tiny mitochondria. These cells can be positive for different stem cells markers like, but not limited to: NANOG, Oct 3, Oct 4, SOX 3, SOX 4, NESTIN, PAX 6, and CD34.

These ImPS can communicate with other mature cells. They have present in their membrane Connexin 43, one of the components of gap junctions, which can provide a direct link between cells under many physiological processes. One such non-limiting exemplary process is to regulate N-cadherin transcription directly. Connexin 43 can control neural crest cell migration in vivo by directly regulating N-cadherin transcription. Such cell migration may be related with tissue growth and tissue regeneration. This protein Connexin 43 can be found between amphibians' and mammals' cells.

The cellular product can be produced by simply removing the blood and fat cells contained in the abdominal fat tissue. Other than physically separating the fat and the blood cells by centrifuge, the cells are rinsed using normal saline. In an embodiment, there is no additional manipulation.

There is no packaging or advertising associated with this procedure. Whether or not the procedure can or should be performed, is solely determined after patient presentation and evaluation by clinical staff.

The product can be physically separated by centrifuging. In an embodiment, no other step is performed before injection into the patient from which the concentrate is drawn.

The cellular product can be injected intravenously into the same patient during the same procedure on the same day.

The presently described processes of removing fat and blood can be performed using a minimally manipulated process described herein, and thus can avoid affecting the intended HCT/P product and thus, is “such HCT/P”.

Accordingly, the present subject matter relates to a method for extracting immature pluripotent stem cells from human fat or human fat tissue, i.e., ImPS.

Once the ImPS are separated from the fat tissue, they can be used with saline to treat patients with disease or injuries. For example, the ImPS can be administered to the patient intravenously or locally.

The Immature Pluripotent Stem Cells (ImPS) as described herein can have many possible functions, including but not limited to the following:
1) The stem cells can arrive to the organ, organs or tissue which are damaged and may need to be repaired to a healthy tissue. Repairing the tissue can allow the organ, organs or tissue to work normally and improve or cure the health condition of the patient, giving a better quality of life.
2) The stem cells can be used in rejuvenation methods to patients who are aging but do not have a clear disease.
3) The stem cells can be used for anti-aging purposes.
4) The stem cells can be used to improve energy in a healthy patient.
5) The stem cells can be used for improving the immune system and avoiding diseases like flu, cold, and infections, among others.
6) The stem cells can be used for patients with cancer to decrease the cancer effects, and/or to improve the immune system before chemotherapy and radiotherapy.
7) The stem cells can be used to improve sexual activity, libido.
8) The stem cells can be used for treating Erectile Dysfunction.
9) The stem cells can be used for decreasing and suppressing pain in areas of joints.
10) The stem cells can be used to decrease pain and perception of pain in patients
11) The stem cells can be used to accelerate healing in skin, muscle, bone, etc.
12) The stem cells can be used to regenerate nerves.
13) The stem cells can be used to improve heart and vascular function.
14) The stem cells can be used to improve kidney function.
15) The stem cells can be used to improve liver function.
16) The stem cells can be used to improve cognitive function.
17) The stem cells can improve brain blood flow.
18) The stem cells can improve heart blood flow.
19) The stem cells can improve leg blood flow.
20) The stem cells can improve penis blood flow.
21) The stem cells can improve blood flow in all organs.
22) The stem cells can alleviate the pain and improve function in patients with fibromyalgia.
23) The stem cells can be used for infertility treatment in women or men, to regenerate the mitochondria and products required for cell metabolism.

The ImPS can be stimulated by all inflamed organs, tissue, cells, etc. They can be attracted to organs, tissue, cells, etc. which secrete some different types of chemokines because these types of stem cells have receptors for these chemokines, and probably other types of genes like IFN-beta, Copaxone, toll like receptors, etc. Once the ImPS are attracted to and get in direct contact with the membrane of the inflamed cells, they can start to communicate throughout some inter-cell membrane connexins (for example: is Cx 43), pass genetic information such as what type of cells are inflamed, and the ImPS can start to differentiate into that specific type of cell. For example, if they are in contact (synapse-like) with heart muscle cells they can differentiate into heart muscle cells. The ImPS can have some characteristics similar to any other type of stem cells. For example, they can start to generate blood vessels at the first step (neovascularization). Physiologically the regenerated tissue may need to have a new blood vessel to improve the tissue's life. The damaged tissue must undergo neovascularization to maintain tissue growth/regeneration with high demands for nutrients, oxygen, new stem cells from the blood circulation, etc.

Accordingly, the ImPS as described herein can have many unique features, including but not limited to the following:
These stem cells are smaller in size (1-10 microns) compared to other described stem cells.
They can be obtained in large quantities from human fat tissue.
No cell culture is required before injection into patients.
They do not need exposure to a cell culture medium, which might create unpredictable antibodies that could cause side effects as well as influence differentiation in patients receiving stem cell treatment.
The cells can express pluripotency markers such as NANOG, OCT 3, OCT 4, NESTIN, and SOX-4, CD34, PAX6, CD146.
These cells can remain inactive or dormant until stimulated by certain genes activated during injury, inflammation, or stress.
These cells are present in all adult human tissues and all types of fat tissue in the human body.
They can be highly resistant to stress, including changes in temperature, pH, and oxygen concentration.
They can be found in significant quantities even in elderly individuals, such as 97-year-olds.
The quantity of these stem cells may be related to a person's health and diet.
They can possess therapeutic potential as they are immature and undifferentiated.
They can demonstrate hypoxia resistance.
They can be capable of crossing the blood-brain barrier and lung capillaries.
They can home to any part of the body and regenerate damaged tissues.
While they may have limited differentiation in vitro, they can differentiate easily in vivo.
These stem cells can self-renew for extended periods within the human body.
Repeated injections may enhance their ability to regenerate and heal damaged tissue.
They can provide both short-term and long-term improvements in treating human diseases.
They can improve and slow down ALS symptoms in patients treated with these stem cells, even up to and after more than 4 years.
They can cure fibromyalgia in some patients.
They can provide a good stem cell treatment to patients whose religion does not allow them to receive any blood transfusions.
It may be beneficial to bank these stem cells in patients who require higher amounts or who are undergoing treatments that deplete the reserve or the ability of the body to regenerate tissues (i.e., post chemotherapy, radiation therapy).

### Procedure

A small amount of adipose tissue is taken (typically about 60 mL), physically separating without the use of chemicals or enzymes. From the adipose tissue, the cellular fraction with small vessels (pericytes and vascular progenitor cells), white blood cells (monocytes/macrophages) and stress-induced stem cells are isolated. The entire process takes part as one same day procedure.
1. Each 10 mL syringe is placed into a Falcon tube for centrifugation.
2. The centrifuge device to be used will be the Infitek Clinical Centrifuge, Low Speed Benchtop, CFG-5BL.
3. Two washing steps, consisting of centrifuging for ten (10) minutes 2× (twice) at 3500 rpm to separate the multiple cells that exist in adipose tissue.
4. From the vascular fraction, the stem cell component is further isolated and diluted in saline for systemic injection. The cellular effects have not been completely elucidated, but it is generally agreed upon that the systemic effects act to reduce overall inflammation in the body, similar to systemic steroids. To this end, it could be most appropriate for patients with systemic illnesses.
5. The final product is administered intravenously into the same patient (autologous use only) during the same procedure on the same day.

A sterile technique is used for all steps; centrifugation, physical separation using a closed syringe, and extraction of the final cellular product. All of the laboratory procedures are performed in a dedicated space following strict sterile protocols. Only one procedure is performed at any given time in a dedicated fume hood and ultraviolet cleaning is done between procedures for a minimum of forty-five (45) minutes. All materials and tools used are sterile and disposable.

### Patient Selection

1. Patients are selected as candidates for the procedure based on clinical diagnosis from the patient's primary care or other referring or treating physician(s).
2. The patient should have a recent (within one month) history and physical, lab work and imaging from the patient's primary care or other referring or treating provider. Those documents are reviewed to determine the patient's eligibility for treatment.
3. Ineligible patients may include those with significant heart disease, active cancer, advanced age, or diminished psychological capacity to understand the procedure.
4. Once the patient is deemed a candidate, the referring provider and the patient are notified, and the patient will be given an appointment.

### Anesthesia

1. Local anesthesia.
2. During the procedure the patient is monitored to control vital signs.

### Post Procedure

The patient is transported to a private recovery area where vital signs and overall comfort are monitored by a registered nurse for about 30 minutes after the procedure. Patients are able to eat and are usually discharged after the approximately 30-minute observation period. Patients are given discharge instructions and follow-up appointment information.

### Follow-Up

Patients are contacted post procedure per standards for this minimally invasive procedure.

### ALS Treatment Results

A group of 6 ALS patients were followed for more than one year. They are treated with Immature Pluripotent Stem Cells via intravenous and intra spinal. The ImPS are obtained through a small liposuction with a local anesthesia. The fat tissue is washed with saline and centrifuged. The ImPS are separated from the tubes. The ALS treatment protocol is based on trying to:
1. Slow the progression of the disease.
2. Regenerate motoneurons and damage of the nerve tissue; and
3. Repair the collateral damage that this disease can create.

In addition to the treatment with ImPS, daily physical therapy is added to the protocols in order to stimulate the nervous system to create and maintain neuro-muscular synapses. A diet for the patients is developed. This anti-inflammatory diet helps the patient maintain body muscle mass and stimulates the nervous system with foods that keep the nervous system alert. The ImPS treatment is repeated on each patient every three to six months. Improvements in the patients' symptoms are seen. The equilibrium, range of the movement, muscle strength, and muscle body mass improves. A deceleration of the progression of the disease is also seen. The patients have reported that the disease is not advancing as rapidly as it was before the stem cell treatment.

In order to have objective data, a dietician and the team closely follows all the anthropometrics measurements, and the body muscle mass compared to the body mass is viewed. Communication is maintained with the physical therapist, who looks closely at the body muscle strength and who can tell if the patient maintains their muscle strength. The ImPS treatment is effective in patients with ALS. The treatment at least slows down the evolution of this disease and keeps the patients in good and stable condition. There is so much to learn and study in this area, and specifically with this type of stem cells. The contribution of the team to this devastating disease has been observed for patients with ALS, which is very significant.

I claim:

1. A method substantially as hereinbefore described.

2. A process substantially as hereinbefore described.

3. A composition substantially as hereinbefore described.
